(19) United States
(12) Patent Application Publication
(10) Pub. No.: US 2026/0091216 A1
(43) Pub. Date: Apr. 2, 2026

(54) MECHANICAL CIRCULATORY SUPPORT DEVICE WITH BIOIMPEDANCE SENSOR

(71) Applicant: Abiomed, Inc., Danvers, MA (US)

(72) Inventors: Michael Cuchiara, Danvers, MA (US); Verena Zscherlich, Aachen (DE); Seth Holdmeyer, Sharonville, OH (US)

(21) Appl. No.: 19/345,192

(22) Filed: Sep. 30, 2025

Related U.S. Application Data
(60) Provisional application No. 63/701,040, filed on Sep. 30, 2024.

(51) Int. Cl. A61M 60/554; A61M 60/174; A61M 60/226; A61M 60/411; A61M 60/585; A61M 60/829; A61M 60/857

(52) U.S. Cl. CPC A61M 60/554; A61M 60/174; A61M 60/226; A61M 60/411; A61M 60/585; A61M 60/829; A61M 60/857; A61M 2205/32; A61M 2205/3303; A61M 2205/3306; A61M 2205/3327; A61M 2205/3379

(57) ABSTRACT

A heart pump configured to be placed in a heart of a patient is described. The heart pump may include an inlet configured to allow blood from the heart to enter the heart pump, an outlet configured to expel blood into the heart, a set of sensors arranged between the inlet and outlet, and at least one processor. The at least one processor may be configured to receive impedance-based signals from the set of sensors, detect a tissue located proximate to the set of sensors based, at least in part, on the impedance-based signals, determine pump information and/or a cardiac function information associated with the heart of the patient based, at least in part, the detected tissue, and output on a user interface, an indication of the pump information and/or the cardiac function information.

### CROSS-REFERENCE TO RELATED APPLICATIONS

This application claims the benefit under 35 U.S.C. § 119(e) to U.S. Provisional Patent Application No. 63/701,040 , filed Sep. 30, 2024, and titled, “MECHANICAL CIRCULATORY SUPPORT DEVICE WITH BIOIMPEDANCE SENSOR,” the entire contents of which is incorporated herein by reference in its entirety.

### TECHNICAL FIELD

This disclosure relates to a mechanical circulatory support device having a bioimpedance sensor.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella® family of devices (Abiomed, Inc., Danvers, MA).

### SUMMARY

Described herein are systems and methods for using a set of sensors arranged on a portion of a mechanical circulatory support device to detect tissue. In some embodiments, impedance measurements sensed by the set of sensors may be used to determine a position of the mechanical circulatory support (MCS) device in a patient's heart by, for example, distinguishing between tissue and blood at one or more locations along the length of the MCS device. In some embodiments, sensor data from the set of sensors may be used to assess a cardiac function, such as blood volume in a particular heart chamber (e.g., the left ventricle).

In some embodiments, a heart pump configured to be placed in a heart of a patient is provided. The heart pump includes an inlet configured to allow blood from the heart to enter the heart pump, an outlet configured to expel blood into the heart, a set of sensors arranged between the inlet and outlet, and at least one processor. The at least one processor is configured to receive impedance-based signals from the set of sensors, detect a tissue located proximate to the set of sensors based, at least in part, on the impedance-based signals, determine pump information and/or a cardiac function information associated with the heart of the patient based, at least in part, the detected tissue, and output on a user interface, an indication of the pump information and/or the cardiac function information.

In one aspect, the heart pump further includes a cannula coupled between the inlet and the outlet, wherein the set of sensors is arranged along a length of the cannula. In another aspect, the set of sensors includes a plurality of sensors arranged in pairs of sensors arranged along the length of the cannula. In another aspect, each of the pairs of sensors includes a first sensor configured to be excited by a current or voltage and a second sensor configured to sense a signal during excitation of the first sensor by the current or voltage. In another aspect, each pair of sensors in the set of sensors includes a gap between the first sensor and the second sensor. In another aspect, the heart pump further includes a controller configured to sequentially excite the first sensor in each of the pairs of sensors. In another aspect, the set of sensors includes a first pair of sensors arranged at a first location along the cannula and a second pair of sensors arranged at a second location along the cannula, a first sensor of the first pair of sensors is configured to be excited by a current or voltage, and a first sensor of the second pair of sensors is configured to sense a signal during excitation of the first sensor of the first pair of sensors by the current or voltage. In another aspect, the set of sensors includes a plurality of electrodes formed on a surface of the cannula. In another aspect, the plurality of electrodes are formed on an outer surface of the cannula. In another aspect, at least one of the plurality of electrodes is formed on an inner surface of the cannula. In another aspect, at least one of the plurality of electrodes is a ring-shaped electrode. In another aspect, the plurality of electrodes comprise gold electrodes. In another aspect, the heart pump further includes a radiopaque marker located on the cannula, the radiopaque marker arranged between a first pair and a second pair of the pairs of sensors. In another aspect, the pairs of sensors includes a single pair of sensors. In another aspect, the pairs of sensors includes at least two pairs of sensors.

In another aspect, the heart pump further includes a controller configured to excite a first sensor in the set of sensors with a current or voltage, and a second sensor in the set of sensors is configured to sense a signal in response to excitation of the first sensor by the current or voltage. In another aspect, the heart pump further includes an impeller arranged between the inlet and the outlet, wherein the set of sensors is arranged between the impeller and the inlet. In another aspect, the at least one processor comprises a microprocessor arranged proximate to the set of sensors. In another aspect, the at least one processor is further configured to determine a type of the detected tissue based, at least in part, on the impedance-based signals. In another aspect, determining a type of the detected tissue comprises determining whether the tissue is blood or complex connective tissue. In another aspect, the complex connective tissue corresponds to a heart valve. In another aspect, the complex connective tissue corresponds to chordae tendinae. In another aspect, the complex connective tissue corresponds to papillary muscles. In another aspect, the at least one processor is further configured to determine a phase shift of at least one of the impedance-based signals, and determining the type of tissue comprises determining the type of tissue based, at least in part, on the phase shift.

In another aspect, determining pump information comprises determining a position of the heart pump within the heart of the patient. In another aspect, the set of sensors includes a plurality of pairs of sensors arranged along a length of the heart pump, and determining a position of the heart pump within the heart of the patient comprises determining the position of the heart pump based, at least in part, on the impedance-based signals sensed from at least one pair of the plurality of pairs of sensors. In another aspect, outputting an indication of the pump information comprises outputting an indication of the position of the heart pump on the user interface. In another aspect, outputting an indication of the position of the heart pump comprises outputting an alarm on the user interface when it is determined that the position of the set of sensors does not span a heart valve of the heart. In another aspect, outputting an indication of the position of the heart pump comprises outputting instructions on the user interface to guide a user to reposition the heart pump. In another aspect, outputting instructions on the user interface to guide a user to reposition the heart pump includes instructions to reposition the heart pump using echocardiography, ultrasound, fluoroscopy, or x-rays. In another aspect, the heart pump further includes at least one pressure sensor configured to sense a pressure in a portion of the heart of a patient, and determining the position of the heart pump is further based, at least in part, on a pressure measurement from the at least one pressure sensor. In another aspect, the at least one pressure sensor comprises an optical pressure sensor.

In another aspect, determining cardiac function information associated with the heart of the patient comprises determining a blood volume in a chamber of the heart. In another aspect, the heart pump is configured to be placed across an aortic valve of the heart of the patient, and determining a blood volume in a chamber of the heart comprises determining a blood volume in a left ventricle of the heart. In another aspect, determining cardiac function information associated with the heart of the patient comprises determining a valvular function of a heart valve. In another aspect, determining a valvular function of the heart valve comprises determining a compliance of the heart valve. In another aspect, determining a valvular function of the heart valve comprises determining a metric associated with opening and/or closing of the heart valve around the heart pump.

In some embodiments, a computer-implemented method is provided. The computer-implemented method includes receiving impedance-based signals from a set of sensors arranged on a heart pump, detecting, using at least one computer processor, a tissue located proximate to the set of sensors based, at least in part, on the impedance-based signals, determining pump information and/or a cardiac function information associated with a heart of a patient within which the heart pump is implanted based, at least in part, the detected tissue, and outputting on a user interface, an indication of the pump information and/or the cardiac function information.

In one aspect, the set of sensors are arranged along a length of a cannula of the heart pump, wherein the cannula is coupled between an inlet of the heart pump and an outlet of the heart pump. In another aspect, the set of sensors includes a plurality of sensors arranged in pairs of sensors arranged along the length of the cannula. In another aspect, each of the pairs of sensors comprises a first sensor and a second sensor, the method further includes exciting the first sensor in each of the pairs of sensors with a current or voltage, and receiving impedance-based signals from the set of sensors comprises receiving the impedance-based signals from the second sensor in each of the pairs of sensors during excitation of the first sensor by the current or voltage. In another aspect, each pair of sensors in the set of sensors includes a gap between the first sensor and the second sensor. In another aspect, the method further includes sequentially exciting the first sensor in each of the pairs of sensors. In another aspect, the set of sensors includes a first pair of sensors arranged at a first location along the cannula and a second pair of sensors arranged at a second location along the cannula, the method further comprises exciting at least one sensor in the first pair of sensors by a current or voltage, and receiving impedance-based signals from the set of sensors comprises receiving the impedance-based signals from at least one sensor in the second pair of sensors during excitation of the at least one sensor in the first pair of sensors by the current or voltage.

In another aspect, the method further includes exciting a first sensor in the set of sensors with a current or voltage, and receiving impedance-based signals from the set of sensors comprises receiving the impedance-based signals from a second sensor configured to sense a signal in response to excitation of the first sensor by the current or voltage. In another aspect, the method further includes determining a type of the detected tissue based, at least in part, on the impedance-based signals, and determining pump information and/or a cardiac function information associated with the heart of the patient is based, at least in part, on the type of the detected tissue. In another aspect, determining a type of the detected tissue comprises determining whether the tissue is blood or complex connective tissue. In another aspect, the complex connective tissue corresponds to a heart valve. In another aspect, the complex connective tissue corresponds to chordae tendinae. In another aspect, the complex connective tissue corresponds to papillary muscles. In another aspect, determining the type of tissue comprises determining the type of tissue based, at least in part, on a phase shift associated with the impedance-based signals.

In another aspect, determining pump information comprises determining a position of the heart pump within the heart of the patient. In another aspect, the set of sensors includes a plurality of pairs of sensors arranged along a length of the heart pump, and determining a position of the heart pump within the heart of the patient comprises determining the position of the heart pump based, at least in part, on the impedance-based signals sensed from at least one pair of the plurality of pairs of sensors. In another aspect, outputting an indication of the pump information comprises outputting an indication of the position of the heart pump on the user interface. In another aspect, outputting an indication of the position of the heart pump comprises outputting an alarm on the user interface when it is determined that the position of the set of sensors does not span a heart valve of the heart. In another aspect, outputting an indication of the position of the heart pump comprises outputting instructions on the user interface to guide a user to reposition the heart pump. In another aspect, outputting instructions on the user interface to guide a user to reposition the heart pump includes instructions to reposition the heart pump using echocardiography, ultrasound, fluoroscopy, or x-rays. In another aspect, determining the position of the heart pump is further based, at least in part, on a pressure measurement from at least one pressure sensor located on the heart pump.

In another aspect, determining cardiac function information associated with the heart of the patient comprises determining a blood volume in a chamber of the heart. In another aspect, the heart pump is configured to be placed across an aortic valve of the heart of the patient, and determining a blood volume in a chamber of the heart comprises determining a blood volume in a left ventricle of the heart. In another aspect, determining cardiac function information associated with the heart of the patient comprises determining a valvular function of a heart valve. In another aspect, determining a valvular function of the heart valve comprises determining a compliance of the heart valve. In another aspect, determining a valvular function of the heart valve comprises determining a metric associated with opening and/or closing of the heart valve around the heart pump.

### BRIEF DESCRIPTION OF DRAWINGS

FIG. 1 shows an illustrative heart pump device, in accordance with some embodiments.

FIG. 2 shows an illustrative placement of a heart pump device within the heart of a patient, in accordance with some embodiments.

FIG. 3 illustrates a difference in impedance sensed for different types of tissues, in accordance with some embodiments.

FIG. 4 schematically shows a set of sensors arranged on a heart pump device, in accordance with some embodiments.

FIG. 5A schematically illustrates an impedance measurement technique using four electrodes, in accordance with some embodiments.

FIG. 5B schematically illustrates an impedance measurement technique using two electrodes, in accordance with some embodiments.

FIG. 5C schematically illustrates an impedance measurement technique using a voltage source to excite a set of electrodes, in accordance with some embodiments.

FIG. 6 is a flowchart of a process for determining the position of a heart pump based on impedance measurements sensed from a set of sensors arranged on a heart pump device, in accordance with some embodiments.

### DETAILED DESCRIPTION

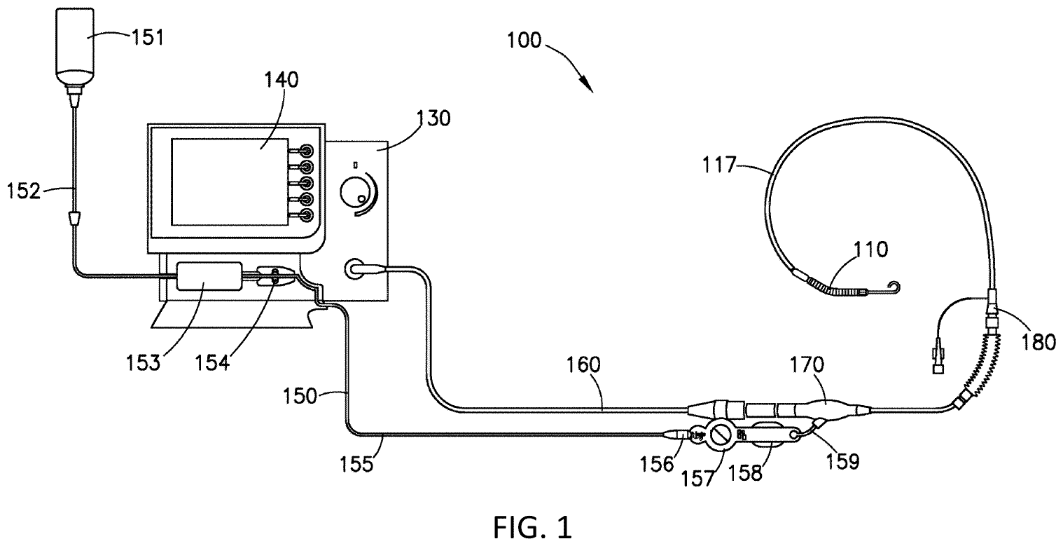

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned in the left side of the heart. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

Proper placement of a heart pump may be important for safety and efficacy of the device during operation. For instance, migration of the pump away from its proper placement (e.g., across the aortic valve for a device placed in the left side of the heart) may result in one or more of reduced pump flow and compromised efficacy, increased hemolysis and poor outcomes for the patient, or a poor user experience, premature removal of the pump, and/or reduced future utilization of the pump. The inventors have recognized and appreciated that existing pump position assessment techniques, which rely only on signals from pressure-based sensors, may be improved by including a set of impedance-based sensors on the heart pump device that may more precisely track the position of the heart pump (e.g., in real-time) during operation of the pump.

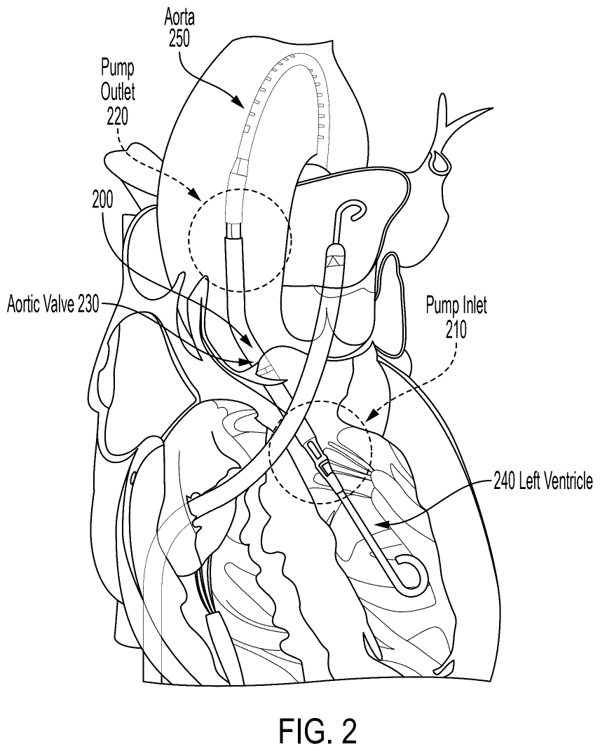

FIG. 2 illustrates an example heart pump 200 when properly positioned in the left side of a patient's heart, in accordance with some embodiments. Heart pump 200 may be an example of heart pump 110 described in connection with cardiac support system 100 shown in FIG. 1. As shown in FIG. 2, heart pump 200 includes an inlet 210 located in the left ventricle 240 of the patient's heart and an outlet 220 located in the aorta 250 of the patient's heart. When in operation, heart pump 200 pumps blood from the left ventricle 240 through the aortic valve 230 and into the aorta 250. In some embodiments, heart pump 200 includes one or more pressure sensors (not shown), which may be configured to measure a pressure near the inlet and/or outlet of the heart pump 200. Pressure signals measured by the one or more pressure sensors may be used, among other things, to estimate a position of the pump in the patient's heart.

Although not labeled, FIG. 2 also shows another heart pump positioned in the right side of the patient's heart. It should be appreciated that although two heart pumps may be used in some patients, other patients may only require the use of a single heart pump placed in the left side of the patient's heart or the right side of the patient's heart. The techniques described herein for determining pump information (e.g., pump position information) and/or cardiac information (e.g., left ventricle blood volume) using a set of impedance-based sensors arranged on the heart pump are applicable to a right-sided heart pump or a left-sided heart pump, and embodiments are not limited in this respect.

As described in connection with the cardiac support system 100, heart pump 200 may be coupled to a controller (e.g., controller 130) configured to control operation (e.g., pump speed, etc.) of heart pump 200. The controller may be configured to receive data (e.g., pump operation data, patient physiological data) from the connected heart pump 200 and an indication of the received data may be displayed on a user interface (e.g., a graphical user interface (GUI)) associated with the controller. In some embodiments, the user interface may include one or more control elements that enable a user to control an operation of the heart pump 200 connected to the controller. For instance, a healthcare provider may interact with one or more of the control element(s) to adjust a pump speed of connected heart pump 200. A display on which the user interface is presented may be integrated with the controller or the display may be provided separate from, but in communication with, the controller.

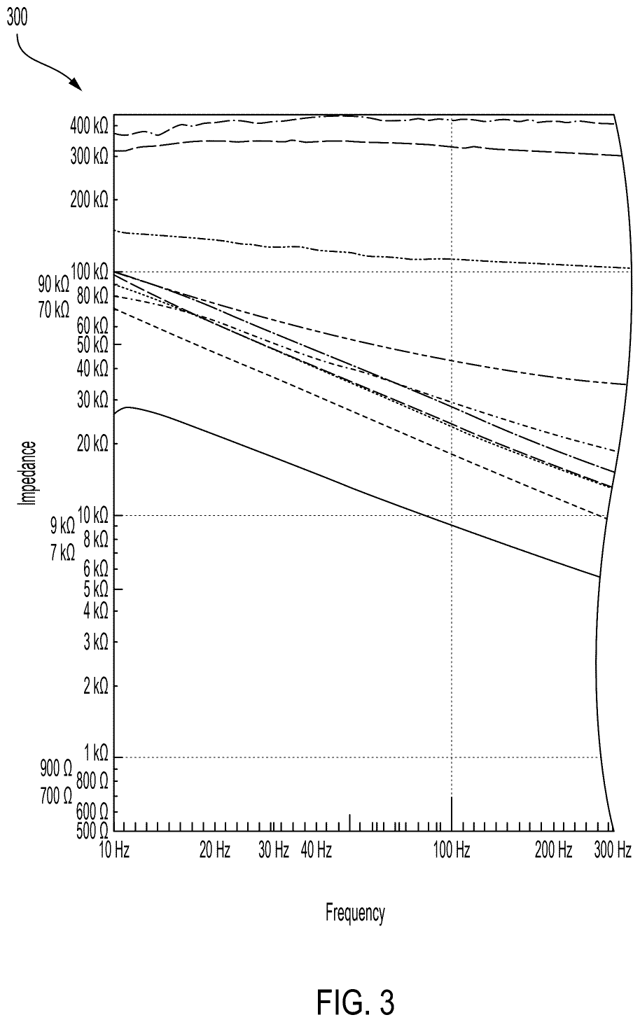

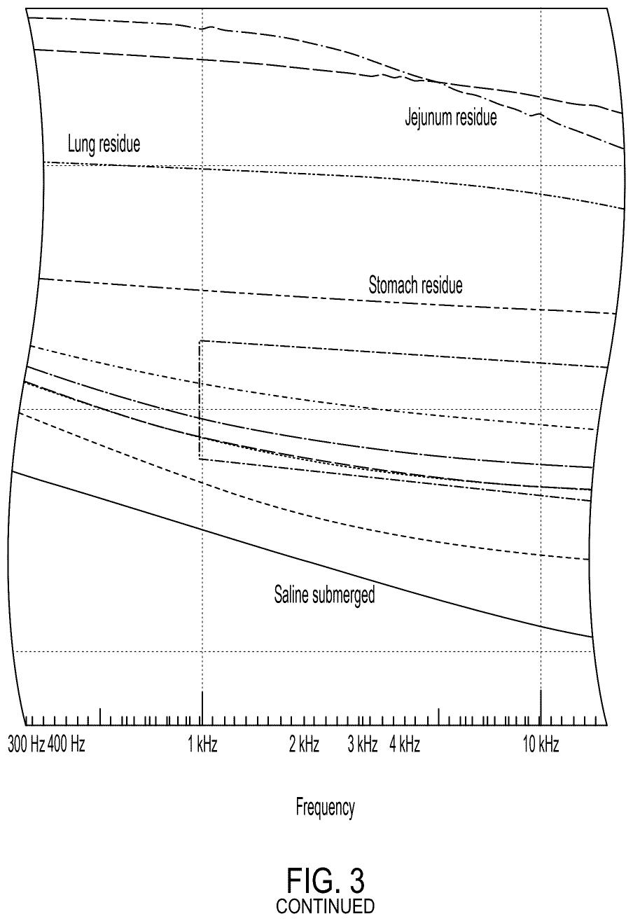

Having the heart pump remain properly positioned in a patient's heart (e.g., across the aortic valve for a left sided device) may be important for effective pump functioning. The inventors have recognized and appreciated that existing techniques for determining pump position (e.g., using only signals from one or more pressure sensors located on the heart pump) may be improved by including a set of impedance-based sensors on the heart pump. FIG. 3 is a plot 300 of impedance vs. frequency showing that different types of biological tissues are associated with different characteristic impedances. Some embodiments leverage these differences in tissue impedance to provide a sensing system on a heart pump that can detect tissue proximate to the sensing system. Such tissue detection can be used, for example, to distinguish between different types of tissue, e.g., complex connective tissue of a heart valve, such as the aortic valve, chordae tendinae, papillary muscles, etc. and blood tissue. By determining that some sensors in the set of sensors are located proximate to blood vs. complex connective tissue, a positioning of the pump relative to a heart valve may be determined with more precision than existing pressure-based sensing techniques. In some embodiments, detecting tissue proximate to the sensing system may include sensing contact with a tissue (e.g., a valve, myocardial or aortic tissue, etc.) and detecting the type of tissue in contact with the sensing system based, at least in part, on an analysis of the duration and time point of the contact (e.g., within the cardiac cycle). For example, information about the cardiac cycle of the patient may be determined based, at least in part, on sensed data from a pressure sensor located on the heart pump, and the cardiac cycle information may be used in combination with impedance information from the sensing system to determine a type of tissue proximate the sensing system. In some embodiments, such impedance-based sensors may additionally or alternatively be used to determine one or more cardiac functions, such as blood volume in a cardiac chamber (e.g., the left ventricle).

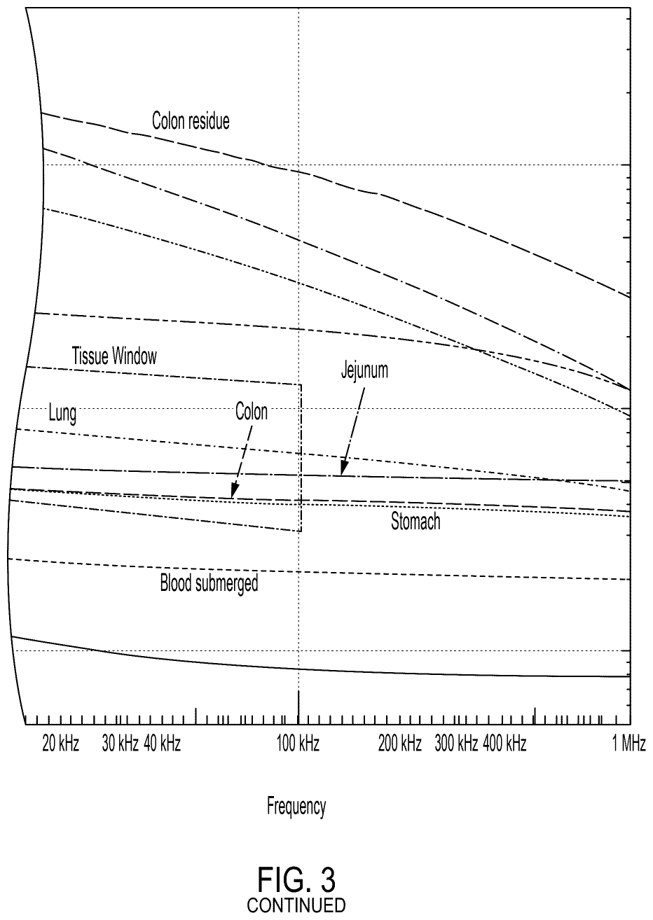

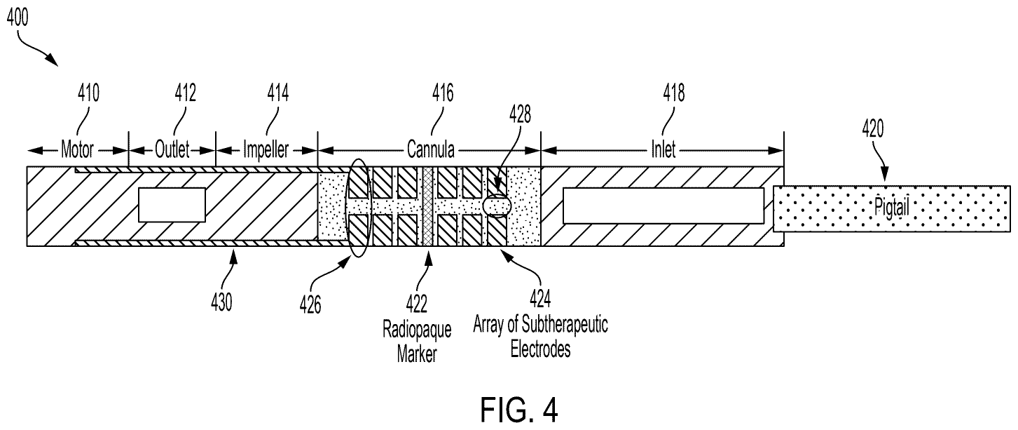

FIG. 4 schematically illustrates a heart pump 400 including a set of sensors 424 (e.g., impedance-based sensors), in accordance with some embodiments of the present disclosure. As shown in FIG. 4, heart pump 400 may include a motor 410 configured to drive rotation of an impeller 414 such that blood is drawn into inlet 418 and expelled from outlet 412 at a desired blood flow rate. Heart pump 400 may include atraumatic extension 420 (also referred to herein as a “pigtail”), which may be configured to assist with initial placement and/or stability of heart pump 400 when inserted in a patient's heart. Coupled between inlet 418 and outlet 412 may be cannula 416 through which blood may flow across a heart valve (e.g., the aortic valve for a left-sided heart pump) when properly positioned in the patient's heart. In some embodiments, the set of sensors 424 is arranged on the cannula 416 between the inlet 418 and the outlet 412. A radiopaque marker 422 (e.g., an echogenic radiopaque marker) may be provided (e.g., printed) on the cannula 416 such that the position of the radiopaque marker 422 can be observed using imaging (e.g., ultrasound).

In some embodiments, one or more sensors in the set of sensors 424 may be located on a portion of heart pump 400 other than the cannula 416. For example, at least one sensor in the set of sensors 424 may be arranged on or near the inlet 418 (e.g., located on a cage surrounding the inlet 418) and/or on or near the outlet 412.

In some embodiments, one or more sensors in the set of sensors 424 may be implemented as a ring-shaped electrode arranged on the exterior or interior of cannula 416. In some embodiments, one or more sensors in the set of sensors 424 may be implemented as a plurality of pairs of sensors arranged along the length of cannula 416 (e.g., on the exterior or interior of cannula 416). Each pair of sensors 426 may include a gap 428 arranged between the first sensor in the pair and the second sensor in the pair. When a biological tissue (e.g., blood or other tissue) is present near the gap of a particular pair of sensors, an electrical (e.g., voltage or current) change related to the characteristic impedance of the biological tissue may be sensed from the pair of sensors. In this way, it may be determined at each pair of sensors 426 in the set of sensors, which type of tissue is located proximate the sensor pair. As the heart pump shifts position by migrating within the patient's heart, the change in position may be detected based, at least in part, on the type of tissue sensed by particular pairs of sensors in the set of sensors. In this way, the signals sensed by the set of sensors may be considered to be “impedance-based” signals regardless of whether the signal being sensed by the pair of sensors is a voltage, a current, or some other electrical quantity.

In some embodiments, at least some first electrodes in the set of sensors may be configured to be driven or “excited” by a current or voltage and at least some second electrodes in the set of sensors may be configured to sense a signal during excitation of the at least some first sensors. The excitation electrode(s) when driven by a voltage or current may emit an electric field, and the sensing electrode(s) may sense a tissue proximate to the sensing electrode(s) by detecting a change in the emitted electric field. In some embodiments, outermost electrodes (e.g., those farthest from the radiopaque marker) may be used as excitation electrodes and inner electrodes (e.g., those closer to the radiopaque marker) may be used as sensing electrodes. In some embodiments, the set of sensors may include radial electrodes (e.g., ring-shaped electrodes) radially arranged around a surface of the cannula at a single location along the length of the cannula. In such embodiments, some of the radial electrodes may be configured to emit an electric field when excited and other of the radial electrodes (e.g., at the same location along the length of the cannula) may be configured as sensing electrodes used to sense a tissue in proximity to the sensing electrodes by detecting a change in the emitted electric field. It should be appreciated that any suitable arrangement of sensors may be used as excitation and sensing electrodes, and embodiments of the present disclosure are not limited in this respect.

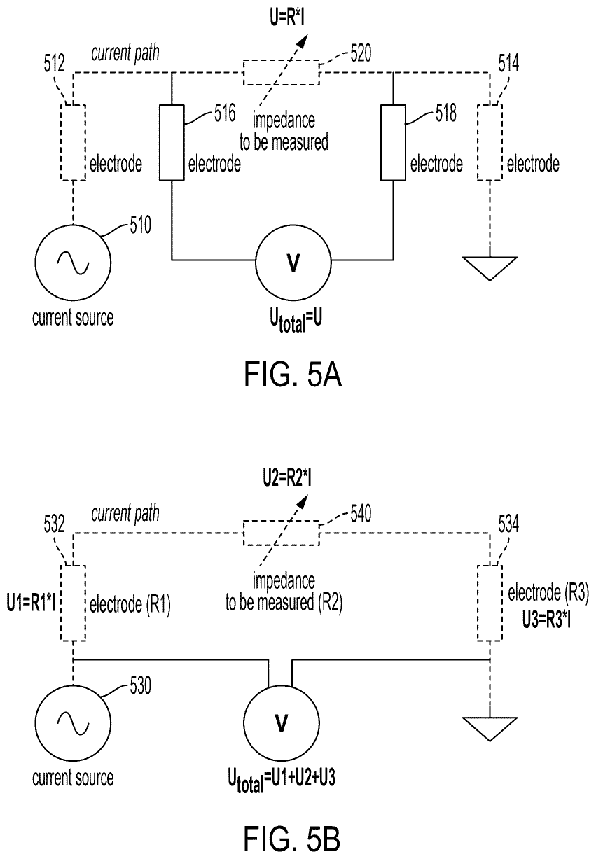

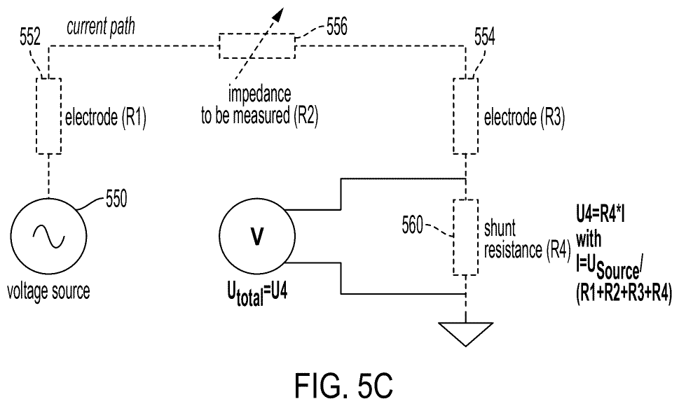

FIG. 5A schematically illustrates an impedance measurement technique in which four sensors are used. As shown, a current source 510 is configured to excite a first electrode 512 and a second electrode 514. Current source 510 may be configured to supply a constant alternating current, and an impedance 520 to be measured may be based on a voltage U sensed across a third electrode 516 and fourth electrode 518. In such a configuration, no (or minimal) current may flow through the third electrode 516 and the fourth electrode 518, configured as sensing electrodes due, at least in part, to the high input resistance of the sensing electrodes. In some embodiments, the third electrode 516 and the fourth electrode 518 may be located on opposite sides of radiopaque marker 422. In some instances, such an implementation may be more robust to signal drifting compared with a two-point measurement technique because changes in the impedance of the sensing electrodes may have no influence on the measurement (provided any changes in impedance are the same for both sensing electrodes, which may occur, for example, due to protein or cell adhesion during use of the heart pump). In some instances, such as when the resistance R to be measured is small compared to the resistance of the electrodes, use of the four-point measurement technique shown in FIG. 5A may have advantages compared with a two-point measurement technique.

In some embodiments, one or more (e.g., all) of first electrode 512, second electrode 514, third electrode 516 and fourth electrode 518 may be implemented as ring-shaped electrodes arranged on a surface of a cannula between the inlet and the outlet of the heart pump. Although the example impedance measurement technique shown in FIG. 5A includes only four electrodes, it should be appreciated that a similar technique may be used with more than four electrodes (e.g., six electrodes, eight electrodes, etc.). For instance, the two outermost electrodes may be used as excitation electrodes and pairs of electrodes equally spaced from and located on opposite sides of radiopaque marker 422 may be used as sensing electrodes.

FIG. 5B schematically illustrates an impedance measurement technique in which two sensors are used. In the example of FIG. 5B, a current source 530 is configured to excite a first electrode 532 and a second electrode 534. An impedance 540 (R2) to be measured across the first electrode 532 and the second electrode 534 may be based on a voltage Uₜₒₜₐₗ measured across the first electrode 532 and the second electrode 534. As shown in FIG. 5B, when using only two electrodes for exciting and sensing, changes in the impedance of the electrodes may have an impact on the sensing measurements. In some embodiments, the first electrode 532 and/or the second electrode 534 may be implemented as small planar electrodes (e.g., as shown in FIG. 4). In some embodiments in which the set of sensors includes pairs of sensors, one or more of the pairs of sensors may be configured to perform a two-point measurement as shown in FIG. 5B. In some embodiments, pairs of sensors including a first sensor located a first position along the length of a cannula and a second sensor located at a second position along the length of the cannula may configured to perform a two-point measurement as shown in FIG. 5B. For instance, adjacent electrodes arranged along the length of the cannula may be used to perform a two-point measurement.

FIG. 5C schematically illustrates an impedance measurement technique in which a voltage source is used to excite sensors rather than a current source as shown in FIGS. 5A and 5B. In the example of FIG. 5C, voltage source 550 may be configured to supply a constant alternative voltage to induce a current in first electrode 552, second electrode 554 and shunt resistance 560, which may be implemented as a resistor having a known resistance. An impedance 556 (R2) to be measured across the first electrode 552 and the second electrode 554 may be determined by measuring a voltage Uₜₒₜₐₗ across shunt resistance 560. As with the impedance measurement technique shown in FIG. 5B, changes in the impedance of the electrodes may have an impact on the sensing measurements due to the excitation current flowing through the same electrodes that are used for sensing. In some embodiments, the first electrode 552 and/or the second electrode 554 may be implemented as small planar electrodes (e.g., as shown in FIG. 4). In some embodiments in which the set of sensors includes pairs of sensors, one or more of the pairs of sensors may be configured to perform a two-point measurement as shown in FIG. 5C. In some embodiments, pairs of sensors including a first sensor located a first position along the length of a cannula and a second sensor located at a second position along the length of the cannula may configured to perform a two-point measurement as shown in FIG. 5C. For instance, adjacent electrodes arranged along the length of the cannula may be used to perform a two-point measurement.

In some embodiments, one or more locations along the length of a cannula of a heart pump device (e.g., cannula 416 of heart pump 400 shown in FIG. 4) may include both excitation and sensing electrodes. For instance, a first pair of sensors (e.g., a radially-spaced pair) at a first location along the length of the cannula may be configured as excitation sensors and a second pair of sensors (e.g., a radially-close pair) may be configured as sensing sensors.

In some embodiments, at least some of the sensors in the set of sensors 424 may be coupled to a controller configured to excite the sensors according to a particular pattern (e.g., according to any of the impedance measurement techniques schematically shown in FIGS. 5A-5C or others). In some embodiments, the controller may excite the pairs of excitation sensors sequentially such that individual pairs of sensors do not interfere with each other when excited. In some embodiments, the set of sensors 424 may include a plurality of electrodes formed of a conducting material such as gold, platinum, or a mixture of conducting materials (e.g., gold with a thin platinum layer on top). For instance, such electrodes may be printed using any suitable deposition technique on the surface of cannula 416.

In some embodiments, a first impedance-based signal sensed from one or more first sensors in the set of sensors may be compared to a second impedance-based signal sensed from one or more second sensors in the set of sensors. For instance, the first impedance-based signal may be used as a reference signal to improve the robustness and/or reliability of the tissue sensing techniques described herein. In some embodiments, at least some (e.g., all) of the sensors 424 may be located on an outer surface of the cannula 416. In some embodiments, at least one of the sensors 424 may be located on an inner surface of the cannula 416. Including at least one of the sensors 424 on an inner surface of the cannula, may be used as a reference sensor configured to continuously contact blood. Such a reference signal may be compared with signals from one or more sensors arranged on an exterior surface of the cannula to improve the tissue detection techniques described herein. In some embodiments, the set of sensors 424 may include a plurality of pairs of sensors with the gaps between each pair of sensors being aligned as shown in FIG. 4. In other embodiments, the gaps between each pair of sensors may be misaligned with adjacent pairs of sensors around the circumference of cannula 416. In some embodiments, one or more of the sensors 424 may be implemented as a ring-shaped sensor.

In some embodiments, signals sensed by the set of sensors 424 may be provided by one or more signal lines to a processor configured to process the signals. In some embodiments, heart pump 400 may include a microprocessor (not shown) located proximate to the set of sensors 424 to perform local processing of the signals sensed by the set of sensors. For instance, the microprocessor may be located within the heart pump assembly itself, within a controller connected to the heart pump assembly, or within a device arranged near the heart pump assembly. In some embodiments, the processor may be configured to determine a type of tissue located proximate to the set of sensors (or a portion of the set of sensors) based, at least in part, on the impedance-based signals received from the set of sensors 424. For instance, the processor may be configured to use information such as that shown in plot 300 of FIG. 3 to distinguish between different types of tissue based on the impedance-based signals received from the set of sensors and each type of tissue's characteristic impedance at a given stimulation frequency. As an example, it may be helpful when assessing a heart pump position to determine whether the detected tissue is blood or complex connective tissue included in a heart valve. In some embodiments, the processor may be configured to determine a type of tissue based, at least in part, on a phase shift of one or more of the impedance-based signals. In some embodiments, the processor may be configured to determine a type of tissue based, at least in part, on information from a secondary sensor located on the heart pump. For instance, information from at least one pressure sensor (e.g., at least one optical sensor), a motor current sensor, or some other sensor located on the heart pump may be used to augment and/or confirm the position determined using the set of sensors 424 located on the cannula 416.

The output of the processor may be provided to a user interface configured to output an indication of pump information (e.g., pump position information) and/or cardiac function information, examples of which are described herein. For instance, the user interface may be configured to display one or more of a visual indication of the position of the pump, an alarm when it is determined that the position of the set of sensors does not span a heart valve of the patient's heart, or instructions guiding a healthcare provider or other user to reposition the heart pump. In the example of providing instructions, the instructions may include instructions to reposition the heart pump using medical imaging examples of which include, but are not limited to, echocardiography, ultrasound, fluoroscopy, or x-ray imaging.

In some embodiments, the processor may be configured to process the impedance-based signals to determine a cardiac function of the heart of the patient. For example, the processor may be configured to determine a blood volume of a chamber of the patient's heart based on the impedance-based signals. When the heart pump is inserted into the left side of the patient's heart, the processor may be configured to determine a blood volume of the left ventricle of the heart. The processor may additionally or alternatively be configured to determine other cardiac functions using the impedance-based signals. For example, in some embodiments, the processor may be configured to determine a valvular function of a heart valve. For instance, the valvular function may relate to how effectively the heart valve is opening and/or closing around the heart pump when inserted through the valve. Opening/closing of the heart valve may be assessed by sensing an impedance change over time as the valve opens and closes around the pump. As another example, the valvular function may relate to a healthiness of the heart valve by detecting aortic valve insufficiency. As another example, valvular function may relate to a compliance of the heart valve. The processor may be additionally or alternatively configured to determine other types of cardiac function information examples of which include, but are not limited to, end diastolic volume, end systolic volume, stroke volume, ejection fraction, cardiac stroke work, pre-load recruitable stroke work, pressure volume area, and arterial elastance. In some embodiments, the cardia function information may be determined using information from the impedance-based signals and other information (e.g., information from a pressure sensor located on the heart pump). An indication of the determined cardiac function information may be provided on a user interface (e.g., a user interface on a console associated with the heart pump) as a numerical value, plot, alarm, or using any other suitable indicator.

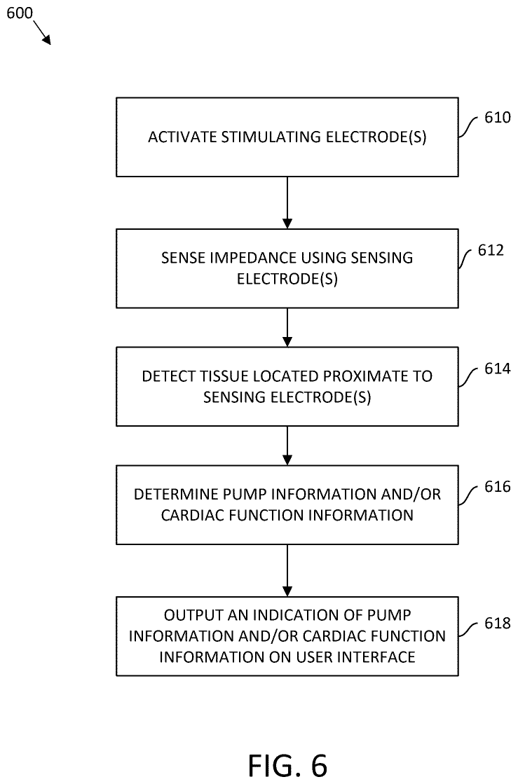

FIG. 6 is a flowchart of a process 600 for determining pump information and/or cardiac function information using a set of impedance-based sensors arranged on a heart pump, in accordance with some embodiments. Process 600 may begin in act 610, where one or more stimulating electrodes in the set of sensors is activated or “excited” by providing a current or voltage to the stimulating electrodes. Process 600 may then proceed to act 612, where an impedance is sensed (e.g., by sensing a voltage, current or some other electrical quantity) using one or more sensing electrodes in the set of set of sensors. It should be appreciated that different arrangements and types of electrodes in the set of sensors may be used for excitation and/or sensing, non-limiting examples of which are shown in FIGS. 5A-5C. For instance, a particular arrangement and/or type of electrode(s) in the set of sensors may be selected depending on the quantity (e.g., pump information or cardiac function information) that is being determined. Process 600 may then proceed to act 614, where a tissue located proximate to the sensing electrode(s) is detected. For instance, as described herein, a processor may process impedance-based signals from one or more sensing electrodes to determine a type of tissue located proximate to the sensing electrode(s). In some embodiments, additional information (e.g., from one or more pressure sensors, motor current sensors, or other heart pump sensors) may be used, at least in part, to determine a type of tissue located proximate to the sensing electrode(s). Process 600 may then proceed to act 616, where pump information and/or cardiac function information is determined based on the impedance-based signals. For instance the position of the pump in the heart of a patient may be determined as pump information. Process 600 may then proceed to act 618, where an indication of the pump information and/or cardiac function information is output on a user interface.

Having thus described several aspects and embodiments of the technology set forth in the disclosure, it is to be appreciated that various alterations, modification, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A heart pump configured to be placed in a heart of a patient, the heart pump, comprising:
an inlet configured to allow blood from the heart to enter the heart pump;
an outlet configured to expel blood into the heart;
a set of sensors arranged between the inlet and outlet; and
at least one processor configured to:
receive impedance-based signals from the set of sensors;
detect a tissue located proximate to the set of sensors based, at least in part, on the impedance-based signals;
determine pump information and/or a cardiac function information associated with the heart of the patient based, at least in part, the detected tissue; and
output on a user interface, an indication of the pump information and/or the cardiac function information.

2. The heart pump of claim 1, further comprising a cannula coupled between the inlet and the outlet, wherein the set of sensors is arranged along a length of the cannula.

3. The heart pump of claim 2, wherein the set of sensors includes a plurality of sensors arranged in pairs of sensors arranged along the length of the cannula.

4. The heart pump of claim 3, wherein each of the pairs of sensors includes a first sensor configured to be excited by a current or voltage and a second sensor configured to sense a signal during excitation of the first sensor by the current or voltage.

5. The heart pump of claim 4, wherein each pair of sensors in the set of sensors includes a gap between the first sensor and the second sensor.

6. The heart pump of claim 4, further comprising a controller configured to sequentially excite the first sensor in each of the pairs of sensors.

7. The heart pump of claim 3, wherein
the set of sensors includes a first pair of sensors arranged at a first location along the cannula and a second pair of sensors arranged at a second location along the cannula,
a first sensor of the first pair of sensors is configured to be excited by a current or voltage, and
a first sensor of the second pair of sensors is configured to sense a signal during excitation of the first sensor of the first pair of sensors by the current or voltage.

8. The heart pump of claim 2, wherein the set of sensors includes a plurality of electrodes formed on a surface of the cannula.

9-12. (canceled)

13. The heart pump of claim 3, further comprising a radiopaque marker located on the cannula, the radiopaque marker arranged between a first pair and a second pair of the pairs of sensors.

14-15. (canceled)

16. The heart pump of claim 1, further comprising a controller configured to excite a first sensor in the set of sensors with a current or voltage,
wherein a second sensor in the set of sensors is configured to sense a signal in response to excitation of the first sensor by the current or voltage.

17-18. (canceled)

19. The heart pump of claim 1, wherein the at least one processor is further configured to determine a type of the detected tissue based, at least in part, on the impedance-based signals.

20-23. (canceled)

24. The heart pump of claim 19, wherein
the at least one processor is further configured to determine a phase shift of at least one of the impedance-based signals, and
determining the type of tissue comprises determining the type of tissue based, at least in part, on the phase shift.

25. The heart pump of claim 1, wherein determining pump information comprises determining a position of the heart pump within the heart of the patient.

26. The heart pump of claim 25, wherein
the set of sensors includes a plurality of pairs of sensors arranged along a length of the heart pump, and
determining a position of the heart pump within the heart of the patient comprises determining the position of the heart pump based, at least in part, on the impedance-based signals sensed from at least one pair of the plurality of pairs of sensors.

27. The heart pump of claim 25, wherein outputting an indication of the pump information comprises outputting an indication of the position of the heart pump on the user interface.

28. The heart pump of claim 27, wherein outputting an indication of the position of the heart pump comprises outputting an alarm on the user interface when it is determined that the position of the set of sensors does not span a heart valve of the heart.

29. The heart pump of claim 28, wherein outputting an indication of the position of the heart pump comprises outputting instructions on the user interface to guide a user to reposition the heart pump.

30. (canceled)

31. The heart pump of claim 26, further comprising at least one pressure sensor configured to sense a pressure in a portion of the heart of a patient, wherein determining the position of the heart pump is further based, at least in part, on a pressure measurement from the at least one pressure sensor.

32. (canceled)

33. The heart pump of claim 1, wherein
the heart pump is configured to be placed across an aortic valve of the heart of the patient, and
determining cardiac function information associated with the heart of the patient comprises determining a blood volume in a left ventricle of the heart.

34-37. (canceled)

38. A computer-implemented method, comprising:
receiving impedance-based signals from a set of sensors arranged on a heart pump;
detecting, using at least one computer processor, a tissue located proximate to the set of sensors based, at least in part, on the impedance-based signals;
determining pump information and/or a cardiac function information associated with a heart of a patient within which the heart pump is implanted based, at least in part, the detected tissue; and
outputting on a user interface, an indication of the pump information and/or the cardiac function information.

39-63. (canceled)
